(19) United States
(12) Patent Application Publication
(10) Pub. No.: US 2020/0138999 A1
(43) Pub. Date: May 7, 2020

(54) Air/Water Purification Ion-Exchange Resin Filtration & UVc Filtration System

(71) Applicant: Amelia Hamilton Cronin, Alexandria, VA (US)

(72) Inventor: Amelia Hamilton Cronin, Alexandria, VA (US)

(21) Appl. No.: 16/656,679

(22) Filed: Oct. 18, 2019

Related U.S. Application Data
(60) Provisional application No. 62/755,596, filed on Nov. 5, 2018.

(51) Int. Cl. A61L 9/20; F24F 3/16

(52) U.S. Cl. CPC A61L 9/20; F24F 2003/1614; F24F 2003/1625; F24F 3/1603

(57) ABSTRACT

A combination of solutions provides unique utility in stopping and/or diminishing pollutants, and in particular acting on biota that source infections including secondary types such as Healthcare Associated Infections or HAIs. Utility solutions include combinations of: UVc light illumination, activated charcoal filtering and adhesion, ion-exchange filtering, and coarse size HEPA filtering, and along with continuous and/or periodic deployment in two UVc light treatment modes to stop and/or reduce physical, chemical and biological pollutant vectors.

### RELATED APPLICATIONS

This application claims priority to and the benefit of U.S. Provisional Patent Application Ser. No. 62/755,596, filed on Nov. 5, 2018 for “Air/Water Purification Ion-Exchange Resin Filtration & UVc Filtration System”, which is fully incorporated here by reference.

### BACKGROUND

One need only observe smog conditions throughout the World to appreciate the varied and profound impacts of air pollution. Air transport brings harmful chemicals, bacteria, fungi, and viral vectors to the indoors, creating significant opportunities for infection and disease. As a side effect of pollution, biological Healthcare Acquired Infections (HAIs) are introduced influencing indoor air quality and human health. The Center for Disease Control (CDC, 2012, Zimlichman et al., 2013) estimates HAIs kill an estimated 99,000-125,000 people annually in the United States and impacting the economy by $95 to $145 billion while spreading malaise and sadness throughout a nominally well-functioning Healthcare system (Markey and Daniel, 2016). All venues can potentially experience similar ramifications of infection and disease, which are partially sourced from air pollution and now supply imminent harm despite best efforts (Leapfrog Group and Castlight Health, 2018 and 2019).

There are many reasons why this infection and disease malaise is so. For example, for over fifty years the use of antibiotics reduced concerns over malingering bacteria as much good was done (O'Neill, 2016). But “super germs” evolved resistance to drugs, as well as resistance to hygienic chemical protocols addressing air and surface sources of disease (Cowan and Lyon, 2019). The feedback loop of biological evolution has supplied only temporary relief from the march of bacterial, fungal, and viral vectors in avoiding cleaning chemicals and antibiotics (O'Neill, 2016, Storr et al., 2017).

Before the advent of antibiotics, ultraviolet light-cleansing techniques were partially effective and potentially reduced the occurrence of humans acquiring additional infections when visiting hospital rooms or clinics. In particular, the healthcare industry used ultraviolet light wavelengths or UVc technologies with a lambda max at approximately 254 nm to varying degrees from the 1930s-1940s to treat tuberculous bacteria in “sanitorium” patients (Cowan, 2016a and 2016b).

Current challenges of pollution, and the introduction of infections, has advanced the need for legacy approaches such as UV technologies as well as new combinations of solutions thereof. According to Dr. Richard Martinello of the Yale School of Medicine, “new healthcare challenges, e.g., multi-drug resistant organisms and Healthcare Acquired Infections (HAIs), can be often be mitigated with UV antimicrobial technologies.” It remains to utilize multiple solutions in combination to stop vector agents of pollution (Cowan and Martinello, 2017).

The combination of solutions here brings great utility to addressing malaises in a variety of venues and scales. Here this utility is applied to indoor air quality of rooms including healthcare examples. This is to bring relief to the contemporary HAI conditions (Velez, 2017) that defies traditional methods of air, room and garment cleaning, as well as infectious vectors from personnel, visitors and patients themselves loading the healthcare environment (Lax et al., 2017, Zimmerman, 2017, CDC, 2019).

The Air/Water Purification Ion-exchange Resin and filters, and UVc reduction system is such a utility combination unit of filtering and resin membrane matrix or layers ion-exchange filtration technologies for air and water vapor that act on impurities. In the case of ambient air, filtration with HEPA or HEPA-like filters and activated charcoal filters are used to extract physical and chemical particulates of more and less than 2.5 microns, with PM2.5 the pollutant particulate size range that is arguably most threatening to human health (EPA, 2012). As the media (e.g., air and/or water vapor gases) are further drawn through the ion-exchange filtration layers, filtration and ion-exchange occurs because different ion resins capture or bond with the pollutants based on their molecular and ionic structure.

Once through the filtration, adhesion and/or ion-exchange processes, the media (air and/or water vapor) is then exposed to UVc light (253-260 nm) thereby causing most bacteria, fungi and/or viruses to be effectively staunched (stopped) and eventually destroyed by their lack of reproduction and the onset of “old age.”

The capabilities of combinations of ion-exchange and other filtration along with UVc light removes the toxic particulates and eliminates the threat of bacteria, fungi, and viruses to infect and promote disease at a molecular level. The output of this Air/Water Purification Ion-exchange Resin and combination physical and chemical UVc filtration system is cleaner and safer air.

The significance of this purification device's potential is notable with respect to pollution at the macro and micro levels, including: at the molecular level, one can extract the air and water pollutants using ion-exchange and by different physical and chemical filtering, such as HEPA-like physical filtering and chemical adhesion filtering with activated charcoal; and then—to ensure the destruction of biota—applying UVc illumination and in this case unique in two treatment modes with one or more units.

### DETAILED DESCRIPTION

The issues of indoor air quality and related infectious agents such as HAIs have been inadequately addressed to date. Previous approaches have focused on single issues rather than the multitude of vectors and various pathways doing harm (Cowan and Lyon, 2018). This approach espouses three broad types of pollutants including the physical, chemical and biological for vectors and their pathways. By addressing each solution in combination over time great relief occurs as judged by examples of Testing and Evaluation and literature search.

Here the solution is addressed in combination.

This utility approach seeks to harness intermolecular forces to separate and reduce pollutant concentrations/counts of harmful vectors in air quality in microscale; Using conventional ion-exchange filters or layers of resins and chemicals that consist of cross-linked polymer matrices and/or layers. Relatively uniform distribution of ion-active sites throughout the structure, which are then cross-layered to capture air or water pollutants is the “macro” part of the molecular solution.

The ion-exchange, activated charcoal and physical (HEPA-like) filters are specifically designed to capture: Volatile Organic Compounds through adhesion such as Formaldehyde and derivatives; Carbon monoxide and dioxide and derivatives; Nitrogen dioxide and derivatives (NOx); Sulfur dioxide and derivatives (SOx); and particulates at various sizes including the PM2.5 micron size range (2.5 microns).

Once in the final unit chamber or layers, the air and/or gaseous water is exposed to the UVc light and is then expelled into the room as cleansed. Continuous operation repeats the processes further staunching room air and new air introduced by HVAC and/or room entry or exit. The UVc light thwarts the biologically active pollutants at the DNA molecular level through dimerization effectively stopping biota by interrupting reproduction.

The use of UVc lighting (bulbs or LEDs) is an additional micro part of the combination as it attacks the DNA of the bacterial or fungal or viral organisms through dimerization of DNA thiamine amino acids.

The practical application is demonstrated by the utility of the indoor air quality in rooms. The unit has dual utility, in a closed housing unit treating air with physical, chemical and biological threat vectors reduced using filtering and reactions in “air mode”.

The utility of the processes is in its long duration and/or continuous use. This promises that the air-borne and gaseous water-phase pollutants will have multiple entrainments by the unit over time. This further drives the staunch and as the room “recruits” new vectors for staunching from air handling/HVAC contributions and inputs from visitors and personnel all over time.

The second utility is to operate the unit in its open housing configuration bathing the room with UVc light to staunch airborne and surficial contaminants in the absence of patient and personnel. The open unit “room illumination mode” acts as an adjunct to air mode, and to traditional and required healthcare room cleaning protocols (e.g., CDC Guideline for Sterilization and Disinfection [2008 and updates 2019] and the Joint Commission or IJC Disinfection and Sterilization [2019], and WHO [Zimmerman, 2017]).

Naturally, the configuration of the unit allows for inclusion of a variety of solutions to augment the combinations to staunch pollutants.

These include a variety of said ionic charge resins or layers that can target physical, chemical and biological “weaknesses” of the target vectors such that the they are filtered, adhered, chemically bonded or exchanged, and/or biologically staunched, and all or mostly removed or reduced in concentration over time.

While addressed here at the scale of a hospital or home room, this application is potentially scalable to larger rooms such as auditoriums or gymnasiums, as well as the case of addressing outdoor scale air quality issues of physical, chemical and biological pollutants.

### Modes

Process combinations are consistent with deploying the unit in two different treatment modes.

### Air Mode

The process of claim 1 wherein that of “air mode” using the closed-unit for staunching air pollutants directed through the combination under continuous or periodic fan-driven operation.

### Room Illumination Mode

The “room illumination mode” treatment is affected wherein the unit is opening via hinges and deployed as a secondary UVc light source during cleaning operations. The UVc lights illuminates room surfaces through the air for about 15 to 30 minutes duration in a 180-degree illumination angle and hemispherical solid angles. This approach augments standardized and required room cleansing procedures (CDC, 2019, IJC 2019) and thereby introduces UVc light throughout the unoccupied room surfaces such as floors, walls, ceilings, as well as “high-touch” furniture, fixtures, equipment, and bathroom surfaces;

The process of “room illumination mode” treatment can be enhanced wherein units can be stacked two or more high in 180-degree, about-face orientation, to staunch unoccupied room surfaces through the air for about 15 to 30 minutes in 2×180-degree angles or approximately 360 degrees spherical solid angles; acting as an isotropic source.

### Time

The element of time was not heretofore recognized as pivotal in the total scheme of things. Previous art focused on one-time, short-term time duration “kill” of biota and time and wattage delivery of power (Boyce, 2016). While a valid approach for one-time treatments and inherently one-dimensional in focus, it does not encompass several facts about treatments. Those realized facts of multi-dimensional contamination include continuous re-admission of pollutants via HVAC and other room air streams, and introduction of pollutants particularly biological examples by human and human activities themselves in rooms; all post-treatment. One-time treatments no matter how powerful and exacting are just that.

### Previous & Current Art

To address these contingencies and to supply multi-dimensional and multi-temporal solutions in a practical unitized format, two room air and surface staunching modes were developed to be employed in a tandem unit at separate times.

Previous and current art thought rooms and surfaces were “clean enough” with one-time illumination “cleaning”.

Time in process exposures for staunching are important and previous art held out that a short-term time duration but powerful exposure would be adequate and at an aperiodic pace. Furthermore, continuous or long duration was deemed adequate for some air stream pollutants; while not addressing other issues such as prolonged human transport of pollutants and propagation on room surfaces and “high touch” surfaces; and rendering no prior art recognition of the need for both acute short-term and chronic long-term time treatments and by a combination of processes, as well as by different timings and geometries of treatment including UVc light treatments. Further, previous art had no or limited combination of processes that address a variety of physical, chemical and biological pollutants; the full combination of processes espoused here including filtration, adhesion, UVc illumination and ion-exchange provide processes to address these pollutants as well as NOx and SOx pollutants now shown to impact births in China (New York Times and Nature Sustainability, 2019).

Previous solutions were partial. This combination and two mode solution is more complete, and unique by virtue of a combination of processes and multi-dimensional and multi-temporal application of treatments.

### EXAMPLES

### Results of Testing and Evaluations

### Chemical-Physical PM2.5/Volatile Organic Compounds (VOC)

Small particulate matter/VOC conditions were examined in twelve different Test “runs” over three months collecting over six hundred PM2.5 measurement readings (Lyon et al. 2019).

The unit device was found to “knock down” PM2.5/VOC “peaks” from highs to lows in a matter of minutes. One criterion for evaluation was the peak measure dropping to approximately 68% (+/− one standard deviation or 68%) of the peak reading and generally this occurred in approximately 15-22 minutes (Appendix A and B, Test1-TestE11, Lyon et al. 2019).

Further testing at two-minute reading intervals (Appendix C1 and C2, TestE12) showed a “knock down” time in finer detail than the 5-minute interval readings (Appendix A, Test1-TestE11, Lyon et al. 2019). The duration of “knock down” using the criterion of +/− one standard deviation and two-minute interval measurements was approximately 12-15 minutes (Appendix C1 and C2, Lyon et al. 2019).

TABLE 1a
Temtop measure of PM 2.5 and reductions by the unit combination of processes.
TestE10, data set

| | | PM2.5 in | | | PM 2.5 in | | | PM 2.5 in |
|---|---|---|---|---|---|---|---|---|
| | | ug/m2 | | | ug/m2 | | | ug/m2 |
| Time in minutes | | Comments | Time in minutes | | Comments | Time in minutes | | Comments |
| [symbol] | [symbol] | [symbol] | [symbol] | 34.2 | | [symbol] | [symbol] | [symbol] |
| [symbol] | [symbol] | [symbol] | [symbol] | 12.8 | | [symbol] | [symbol] | [symbol] |
| [symbol] | [symbol] | | [symbol] | 13.9 | | [symbol] | 36.5 | |
| [symbol] | [symbol] | [symbol] | [symbol] | 34.2 | [symbol] | [symbol] | 27.2 | |
| [symbol] | [symbol] | | [symbol] | 18. [symbol] | [symbol] | [symbol] | [symbol] | |
| [symbol] | [symbol] | [symbol] | [symbol] | [symbol] | | 1310 | 21.7 | [symbol] |
| [symbol] | [symbol] | | [symbol] | [symbol] | | [symbol] | [symbol] | |
| [symbol] | [symbol] | [symbol] | [symbol] | [symbol] | | [symbol] | 19.6 | |
| [symbol] | 77.8 | [symbol] | [symbol] | 21.2 | | 1325 | 12.8 | |
| [symbol] | 44.1 | | [symbol] | [symbol] | | [symbol] | [symbol] | [symbol] |
| [symbol] | [symbol] | | [symbol] | [symbol] | | [symbol] | [symbol] | |
| [symbol] | [symbol] | [symbol] | [symbol] | [symbol] | | [symbol] | 78.3 | |
| [symbol] | [symbol] | [symbol] | [symbol] | [symbol] | [symbol] | 1345 | 22.4 | |
| [symbol] | [symbol] | | | [symbol] | | [symbol] | [symbol] | |
| [symbol] | [symbol] | | [symbol] | 84.6 | [symbol] | [symbol] | [symbol] | |
| [symbol] | [symbol] | | [symbol] | [symbol] | | [symbol] | [symbol] | [symbol] |
| [symbol] | [symbol] | [symbol] | [symbol] | 28.8 | | [symbol] | 67.2 | |
| [symbol] | 44.0 | | [symbol] | 24.3 | [symbol] | [symbol] | [symbol] | |
| [symbol] | [symbol] | | [symbol] | 32.5 | | [symbol] | 23.7 | |
| [symbol] | [symbol] | | [symbol] | [symbol] | | [symbol] | 27.5 | |
| [symbol] | [symbol] | | [symbol] | [symbol] | [symbol] | [symbol] | [symbol] | |
| [symbol] | [symbol] | | [symbol] | [symbol] | | [symbol] | 22.3 | |
| [symbol] | [symbol] | | [symbol] | 32.1 | | [symbol] | 18.3 | |
| [symbol] | [symbol] | | [symbol] | [symbol] | | [symbol] | [symbol] | [symbol] |
| [symbol] | [symbol] | | [symbol] | 22.3 | | [symbol] | [symbol] | |
| [symbol] | [symbol] | [symbol] | [symbol] | 21.0 | | [symbol] | [symbol] | |
| [symbol] | [symbol] | | [symbol] | [symbol] | | [symbol] | [symbol] | |
| [symbol] | [symbol] | | [symbol] | [symbol] | | [symbol] | [symbol] | [symbol] |
| [symbol] | 36.2 | [symbol] | [symbol] | 12.4 | | | | |

| | | | |
|---|---|---|---|
| n = 86, 430 minutes | Average [symbol] 30.# [symbol] | Standard Deviation [symbol] 22.61048841 | Adjacent [symbol] |

[symbol]

indicates data missing or illegible when filed

### Biological

In eleven separate Test “runs” there was a total sample size of n=37 with 28 of 37 Petri dishes germinating growths. 12 of the 37 plates were “down” inside the Device and 9 Petri dishes germinated no or zero growths. These 12 were all inside the unit device filter tower or housing “down” next to the UVc lamp.

Of the 3 Petri dishes in the “down” position that experienced growths, the growths numbered 14, 2 and 1. The ratio of these “down” growths to positions “nearby” and/or “top” and/or “far door” or “far-far” position examples around the room was 14:93+:72+ (Test AA), 2:30+:39+ (Test BB) and 1:15:7 (Test AAA) respectively. Hence, the three growth dishes “down” inside the Device housing were thought to be contaminations both by observations of protocol (“jarring” and 14 small growths) and by relative number ratios just above.

Statistical comparison of the 12 dishes “down” inside the unit device and those 27 outside positions showed a difference in (lack of) growths between 99.9% (+/−3 standard deviations) to 99.99% (+/−4 standard deviations) and beyond.

TABLE 2
Summary Table of run results of counts and statistics for unit combination operations
Tests with growths found by exposure position and statistics ny Test run (rows)

| | Down | | | Far Far | | | | |
|---|---|---|---|---|---|---|---|---|
| | position | Nearby | | across | Device | | | |
| Trial | inside | or adjacent | Far door | the room | top outside | Sample | | Standard |
| name | Device | position | position | position | position | size | Mean | Deviation |
| A | 0 | 1+ | 4 | 3 | | 4 | 2 | 1.58113883 |
| B | 0 | 3 | 1 | | | 3 | 1.333 | 1.242219129 |
| C | 0, 0 | | 11+ | | | 0 | 3.667 | 6.185449729 |
| AA | 14 | 93+ | 22+ | | 23+ | 4 | 63 | 29.50423698 |
| BB | 2 | | 30+ | | 39+ | 3 | 23.667 | 15.75506973 |
| CC | 0 | | | 3 | 4 | 3 | 2.339 | 1.699673171 |
| AAA | 1 | 15 | 7 | 7 | | 4 | 7.5 | 4.974937135 |
| BBB | 0 | 4 | 1 | | | 3 | 1.667 | 1.677673171 |
| CCC | 0 | 17 | 17 | | | 3 | 11.333 | 8.013876853 |
| AAAA | 0 | 400+ | 26+ | 155+ | | 4 | 145.25 | 159.3593619 |
| BBBB | 0 | 400+ | | 400+ | | 3 | 266.667 | 188.5618033 |
| Sample size total | 32 | 8 | 9 | 5 | 3 | 37 | | |

### Discussion

### Chemical-Physical PM2.5/VOC

Twelve Tests with over six hundred PM2.5 observations demonstrated unit capabilities in reducing or “knocking down” a range of levels of pollutants. These included concentrations as high as 150-250 ug/m³ range in concentration and/or the “very unhealthful” level to the 250-400+ ug/m³ or “hazardous” ug/m³ PM2.5 in range as human health conditions characterized by EPA (Appendix A and B, Test1-TestE11, Lyon et al. 2019). The unit also acted on reducing levels of modest affect including the 55-150 ug/m³ “unhealthy” medium range levels and low ranges such as 0-12 ug/m³ “good” and 12-35 ug/m³ “moderate” and 35-55 ug/m³ “unhealthy for sensitive groups” ug/m³ (EPA, 2010, https://www.epa.gov/sites/production/files/2016-04/documents/2012_aqi_factsheet.pdf, and Cronin 2019).

### Biological Petri Dish Exposure and Growths

Analyses of Petri dish growths and non-growths showed a tremendous level of reducing or stopping biota by the UVc light and unit. 9 of 12 dishes “down” inside the housing had no growths whatsoever while the remaining 25 dishes outside the Device in four different positions all showed growths. This result was in 12 different Test “runs” of three to four dishes exposed on different dates and exposures of generally three hours in duration.

### Methods and Materials

### UVc

A UV lamp sold as Green UV model “G500 UV Light Air Purifier for A/C Ducts” 12V with a 14″ bulb and magnetic mount was employed. The listed power was 17 watts. The wavelength of maximum output was approximately 254 nanometers or “nm” or Lambda Max of 254 nm. Much literature research was evaluated to select wavelengths, lamp types and configuration and output characteristics (Appendix K including LightSource 2019 and Philips 2019, Lyon et al. 2019). 254 nm wavelength spectra created by mercury lamps is generally the accepted approach to staunching biota (e.g., Cowan references).

### Housing and Fan

An 8 Inch 750 Cubic Feet per Minute (CFM) “Duct Inline Fan Blower” was employed to entrain, transport and filter, and exhaust treated gaseous-liquid phase air and components to the room by VIVOSUN and Zenhydro (2019). All Tests were run at the 750 CFM rated speed.

### Chemical-Physical

### TemTop

Air quality levels were measured by lasing the air using a Temtop instrument. The model was: TemTop LKC-1000E Air Quality Detector for formaldehyde (HCHO)/PM2.5 Particles/AQI Accurate Testing Air Quality Detector.

Measurements of interest were fine sized particulate matter (PM) approximately 2.5 micrometers or “um” in size which is quite small and being close to the order of the wavelengths of light energy. These size ranges include very fine physical particulates as well as gaseous-liquid phases organic chemical compounds or Volatile Organic Compounds (VOCs).

### Fire

A wood burning fireplace and chimney was used to test and “stress test” unit device capabilities as measured by the Temtop instrument by PM2.5 levels (Appendix C3, Lyon et al. 2019).

The fire would periodically produce a “cold fire” event and hence incomplete combustion of wood products and organic components. This in turn generated “peaks” of PM2.5 above and beyond ambient or background levels.

Over time it was determined the opening of the fireplace door would result in combustion products entering the room rather than fully exiting via the chimney. Often this would occur when the operator was “stoking” or manipulating the fire to burn to a higher degree or to burn “hotter” yielding more compete combustion. While stoking or adding wood logs the door would be open for approximately one to two minutes with a correlated released in combustion products into the room. During these periods the device would work to “knock down” the organic combustion products reducing the PM2.5 readings. (During these periods the operator would exit the room and returning to take readings at the proper time interval.)

The fire would be initiated with a variety of kindling types. These could include small wood fragments, paper, kindling products such as “fatwood”, and ignition sources such as plastic-body lighters.

### Dusting and Sweeping

To understand the effects of particulate matter from a non-fire origin, several tests involved dusting the room with paper towels and sweeping the room with a broom and dustpan (TestE9 Appendix A, Lyon et al. 2019).

### Calculations

Calculations of UVc light on a given targeted volume and area (“aperture”) were made using standard and accepted practices espoused by authoritative references (Bolton and Linden 2003, Chapter 13 in Ward et al. 2018). Details on calculations are provided in Appendix G of Lyon et al. 2019.

The UVc lamp was rated at 17watts by the manufacturer. As an assumed isotropic light source with this Intensity or “I” wattage was projected into the air space with a solid angle (volumetric solid angle omega or Ω) of π steradians. This yielded an active Intensity of approximately 5.4 W per steradian (or J/sec.steradian). This agrees with similar lamps and designed wattage vs active wattage by several other manufacturers (LightSource 2019, Philips 2019).

### Biological

### Petri Dishes

Pre-poured, sterilized Luria-Bertani or Luria broth LB-agar dishes from EZ BioResearch (www.ezbioresearch.com) were used. These had the advantage of being school- and/or habitat-safe and capable of growing “environmental” biota rather than potentially human-unsafe biota. The dishes were delivered to the Test site and refrigerated until a given test and exposure time.

Five horizontal positions within the room were used to use to exposed Petri dishes. These were: down inside the unit device housing or the “down” position, outside near the top of the housing at the exhaust end or “top” position, nearby or adjacent the device housing on the table or “adjacent” position, across the side of the room to the east near a door or “far door” position and far across the room to the south or “far-far” position.

### Incubator

According to suggestions (www.ezbioresearch.com) a styrofoam incubator was constructed and equipped with heating source (40 W incandescent bulb) to maintain a desirable incubation temperature range of 80-100 degrees Fahrenheit. A temperature probe was used for measurement and the top of the incubator was manipulated to keep the temperature within range and not to exceed 100° F. The temperature was usually maintained “around the clock” from 90-94 degrees using an AcuRite digital thermometer with probe.

Incubation was for three to six days depending on ambient room temperature conditions with the dishes inverted to re-capture moisture. Relative incubator moisture was maintained by small, open containers filled with distilled water and replenished as needed.

Growths were counted using a standard grid drawn on paper and placed beneath the dish. This was done for counting purposes and to help image the dish growths if present and the grid providing scale and reference. Growths when present exhibited a range between 0 and 400+ clonal or point growths. No attempt was made to speciate the growths which were likely four species types found over the course of testing.

After counting the dishes were imaged and results stored. Resulting dishes were cooled with refrigeration then frozen and stored in a freezer to form an archive or voucher samples for later evaluation if needed.

### Room Characteristics

To mimic unit device performance in a “real world” situation a large room was selected and used for most all Tests. The large room was approximately 4,132 ft³ in area with three doors and two of which were normally closed for Test operations. Tests were setup with a table for the device, and the Temtop measuring instrument in PM2.5 measurement mode approximately one foot away from the Device housing and approximately two feet below the exhaust (the so-called “adjacent” or “nearby” position). There was an overhead room fan constantly running during the period of experiments. Across the room to the south was the large fireplace. The relative positions of unit device, Temtop, table, and fireplace were maintained throughout the tests and between each Test.

### Counts

Where possible individual growths were counted as “points”. When the counts were in excess of capability to count discrete numbers, the total was recorded as “400+” or “39+”. The “+” notation was not used in statistical analyses but rather to signal abundance. There was also the presence of medium and large growths without “point” or “spot” characteristics. These large or medium area were listed as a growth by estimated surface area within the dish and recorded in the matrix presented here as Table 2 (from Appendix E slides 3-5 for conditions, counts and statistics, and other image slides of the actual Petri dishes, Lyon et al. 2019). Surface area coverage estimates were not included in the statistical analyses due to the nature of mixed measurement units.

### Statistics

Statistics were calculated using Excel files and Excel statistical functions AVERAGE, STDEV.P, MAX and MIN for determining the average or mean, the standard deviation for the population, and maximum and minimum values, respectively.

Parametric or “bell-shaped curve” distribution calculations were made. While the distributions may depart from the normal distribution assumption, these statistics are good indicators. Later, non-parametric tests may be employed to see if there are important departures.

Système international (SI units) and US Customary units were used interchangeably and as appropriate. Where practicable both are listed and conversions are approximate.

### DEFINITIONS

“Knock down”, unit operation whereby PM2.5/VOC “peaks” are reduced in concentration from highs to lows in a matter of minutes.

Pollutants, harmful or potentially harmful volatile organic compounds (VOC), particulate matter (PM), pollutant ionic forms (SOx, NOx), and biotic forms such as bacteria, viruses, and fungi.

Staunching, stopping or halting, diminution, reduction and/or elimination of volatile organic compounds, particulate matter, pollutant ion forms, and biotic forms such as bacteria, viruses, and fungi.

Vectors, pollution agents that can transport and harbor sources of infection and disease.

### REFERENCES

Bolton, J. and Linden, K. 2003. Standardization of methods for fluence (UV dose) determination in benchscale UV experiments, J. Environ. Engr., 129(3), 209-215.

Boyce, J. 2016. Modern technologies for improving cleaning and disinfection of environmental surfaces in hospitals, Antimicrobial Resistance & Infection Control 5:10, Open Access, https://aricjournal.biomedcentral.com/articles/10.1186/s13756-016-0111-x or https://doi.org/10.1186/s13756-016-0111-x

Centers for Disease Control and Prevention (CDC). 2018. HAI Data and Statistics. Centers for Disease Control and Prevention (CDC). http://www.cdc.gov/hai/surveillance/index.html, last accessed Oct. 8, 2019.

Centers for Disease Control and Prevention (CDC). 2019. Options for evaluating environmental cleaning. https://www.cdc.gov/HAI/toolkits/Appendices-Evaluating-Environ-Cleaning.html#b, last accessed Oct. 8, 2019.

Cowan, T. 2016a. Letter to the Editor “Need for Uniform Standards Covering UV-C Based Antimicrobial Disinfection Devices,” Infection Control and Hospital Epidemiology, Vol. 37(8): 1000-1001.

Cowan, T. 2016b. UV Antimicrobial Devices Used to Combat HAIs in Medical Facilities: Is There a Need to Establish Voluntary Industry Efficacy Standards for Their Use? IUVA News, Vol. 18(4): 4-8. http://iuvanews.com/stories/122716/uv-antimicrobial-devices-used-combat-hais.shtml, accessed Jun. 14, 2019.

Cowan, T. and Martinello, R. 2017. Panel Discussion: Fighting HAIs and MDROs with UV-C using Industry, Health Care and Federal Collaboration. IUVA News 19(3), 26-27. http://iuvanews.com/stories/pdf/summer1.pdf, last accessed Oct. 8, 2019.

Cowan, T. and Lyon, J. 2018. An Update from the Yale Healthcare/UV Workshop: Developments in IUVA's Initiative a UV Disinfection Efficacy Standard. Vol.20, Issue 4, p.5-10, https://iuvanews.com/stories/pdf/IUVA_2018_Quarter4-Cowan2.pdf, last accessed Oct. 8, 2019.

Cronin, A. 2019. Mealy's Science Olympiad. Report, 20 p.

EPA. 2010. EPA Needs to Assure Effectiveness of Antimicrobial Pesticide Products, OIG Report No. 11-P-0029. USEPA, Washington, DC.

EPA. 2012. Revised air quality standards for particle pollution and updates to the air quality index (AQI). https://www.epa.gov/sites/production/files/2016-04/documents/2012_aqi_factsheet.pdf, last accessed Aug. 5, 2019. Joint Commission. 2019. Disinfection and Sterilization. https://www.jointcommission.org/topics/hai_portal_disinfection_sterilization.aspx, last accessed Oct. 8, 2019.

Lax, S., Sangwan, N., Smith, D., Larsen, P., Handley, K., Richardson, M., Guyton, K., Krezalek, M., Shogan, B., Defazio, J., Flemming, I., Shakhsheer, B., Weber, S., Landon, E., Garcia-Houchins, S., Siegel, J., Alverdy, J., Knight, R., Stephens, B., and Gilbert, J. 2017. Bacterial Colonization and Succession in a Newly Opened Hospital. Science Translational Medicine, May 24, Vol. 9, Issue 391, eaah6500, DOI: 10.1126/scitranslmed.aah6500 http://stm.sciencemag.org/content/9/391/eaah6500, last accessed Jun. 14, 2019.

Leapfrog Group and Castlight Health. 2018 and 2019. Reports on the 2018 Leapfrog Hospital Survey. http://www.leapfroggroup.org/ratings-reports/reports-hospital-performance, and 2019https://www.leapfroggroup.org/node/1064, last accessed Jun. 16, 2019.

LightSources. 2019. Connecticut, https://www.light-sources.com/, accessed Jun. 14, 2019.

Lyon, J., Reiser, A., Cronin, A., Lyon, B., and Cowan, T. 2019. Device Influences on Indoor Air Quality: Report of Device Testing and Evaluation. 14p. and Appendices A through K.

Philips. 2019. The Netherlands, http://www.lighting.philips.com/main/products/special-lighting/uv-purification.

Markey, M. and M. Daniel. 2016. Medical Error—the Third Leading Cause of Death in the US. BMJ 353:i2139. http://www.bmj.com/content/353/bmj.i2139, last accessed Jun. 14, 2019.

New York Times and Nature Sustainability, 2019. Air Pollution Is Linked to Miscarriages in China, Study Finds: growing evidence of the negative health effects of air pollution on pregnant women and their fetuses. https://www.nytimes.com/2019/10/14/world/asia/china-air-pollution-miscarriages-study.html, and Zhang et al, 2019. Air pollution-induced missed abortion risk for pregnancies, last checked Oct. 15, 2019.

O'Neill, J. 2016. Tackling Drug-Resistant Infections Globally: Final Report and Recommendations the Review on Antimicrobial Resistance. Review on Antimicrobial Resistance, United Kingdom Government, May, 84p.

Storr, J., Twyman, A., Zingg, W., Damani, N., Kilpatrick, C., Reilly, J., Price, L., Egger, M., Grayson, M., Kelley, E., Allegranzi, B., and the WHO Guidelines Development Group. 2017. Core Components for Effective Infection Prevention and Control Programmes: New WHO Evidence-Based Recommendations. Antimicrobial Resistance and Infection Control, Vol. 6:6, http://aricjournal.biomedcentral.com/articles/10.1186/s13756-016-0149-9, last accessed Jun. 20, 2019.

VIVOSUN and Zenhydro. 2019. https://www.ebay.com/itm/iPower-8-Inch-Inline-Duct-Ventilation-Fan-HVAC-Exhaust-Blower-for-Grow-Tent/183897871420?hash=item2ad12ad43c:g:Fw4AAOSwkYZdORwR, https://www.ebay.com/itm/4-6-8-Inline-Duct-Fan-Speed-Control-with-Air-Carbon-Filter-Greenhouse -Combo/142884561841?hash=item21449563b1:m:mrkDEgRtkJUU3ymxOoVKzzA&var=4418697 76652.

Velez, K. 2017. Op-Ed: New Technology Helps Hospitals Protect Patients from Deadly Infections, Health care facilities add ultraviolet light (UV-C) technology to their cleaning and disinfecting regimens. US News & World Report, June 28, https://www.usnews.com/news/healthcare-of-tomorrow/articles/2017-06-28/op-ed-new-technology-helps-hospitals-protect-patients-from-deadly-infections, last accessed Jun. 20, 2019.

Ward, A., Trimble, S., Burckhard, S. and Lyon, J. 2018. Environmental Hydrology. Chapter 13 in the Third Edition. Boca Raton, Fla.

Zimlichman, E., Henderson, D., Tamir, O., Franz, C., Song, P., Yamin, C., Keohane, C., Denham, C., and Bates, D. 2013. Health Care—Associated Infections, A Meta-analysis of Costs and Financial Impact on the US Health Care System. JAMA Intern Med., 2013;173(22):2039-2046. doi:10.1001/jamainternmed.2013.9763, http://jamanetwork.com/journals/jamainternalmedicine/fullarticle/1733452, last accessed Oct. 8, 2019.

Zimmerman, B. 2017. WHO's 8 Guidelines for Effective Infection Prevention Programs. Beckershospitalreview.com, January 11, http://www.beckershospitalreview.com/quality/who-s-8-guidelines-for-effective-infection-prevention-programs.html, last accessed Jun. 15, 2019.

1. A process for reducing pollutants in room air which comprises; directing a stream of room air through a combination of filtering, adhesion, ion-exchange, and UVc light disruption steps, and directing said pollution-reduced room air back into the room.

2. The process of claim 1 wherein a series of four sub-process steps act on room air to: remove course particles through paper or HEPA filtering, chemically adhere volatile organic compounds and finer particles through activated charcoal, exchange anion and cation air pollutants using an ion-exchange layer, and use of UVc illumination to reducing and inactivating biological and chemical pollutants.
